(19) United States
(12) Patent Application Publication
(10) Pub. No.: US 2004/0014797 A1
(43) Pub. Date: Jan. 22, 2004

(54) Pharmaceutical composition comprising metformin and a 5-phenoxyalkyl-2,4-thiazolidinedione-type derivative

(76) Inventors: Gerard Moinet, Orsay (FR); Gerard Botton, Buc (FR); Didier Mesangeau, Combs La Ville (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/343,609

(22) PCT Filed: Jul. 24, 2001
(86) PCT No.: PCT/EP01/08512

(30) Foreign Application Priority Data
Aug. 4, 2000 (FR) 00/10362

(51) Int. Cl. A61K 31/426; A61K 31/155

(52) U.S. Cl. 514/369; 514/635

(57) ABSTRACT

The present invention relates to a pharmaceutical composition comprising, as active ingredients, metformin optionally in the form of one of its pharmaceutically acceptable salts and a 5-phenoxyalkyl-2,4-thiazolidinedione-type derivative.

The present invention relates to a pharmaceutical composition comprising, as active ingredients, metformin optionally in the form of one of its pharmaceutically acceptable salts and a 5-phenoxyalkyl-2,4-thiazolidinedione-type derivative described in WO 97/47612.

Its subject is also the use of metformin optionally in the form of one of its pharmaceutically acceptable salts and a 5-phenoxyalkyl-2,4-thiazolidinedione-type derivative for the preparation of a medicinal preparation intended to reduce hyperglycaemia, more particularly hyperglycaemia in non-insulin-dependent diabetes.

Metformin is mainly known for its antihyperglycaemic activity and is widely used in the treatment of non-insulin-dependent diabetes. In the case of non-insulin-dependent diabetes, metformin is also administered to the patient in combination with insulin, metformin being known to improve sensitivity to insulin.

Numerous 2,4-thiazolidinedione derivatives have been described as antihyperglycaemic and hypolipemic agents and thus described as antidiabetic agents (Takeda, patent EP 193 256 and Sankyo patent EP 207 581). These compounds are activators of the peroxisome proliferator activated receptor γ (PPARγ).

The combination of some 2,4-thiazolidinedione derivatives and biguanide, more particularly metformin, for treating diabetes has already been described (Takeda, patent application EP 749 751 and Smithkline Beecham, patent application WO 98/57634).

Diabetes is a chronic disease exhibiting various pathological manifestations. It is accompanied by disorders in the metabolism of lipids and sugars, and by circulatory disorders. In many cases, diabetes tends to progress into various pathological complications. Thus, it is necessary to find the treatment adapted to each individual suffering from diabetes.

The specific combination of metformin optionally in the form of one of its pharmaceutically acceptable salts with 5-phenoxyalkyl-2,4-thiazolidinedione which has no activity on the transactivation of PPARγ has not been described and offers particular advantages, in particular the absence of weight gain and/or of haemodilution.

Thus, the aim of the present invention is to provide a composition which makes it possible to significantly improve the use of glucose.

Its aim is also to provide a composition suitable for the treatment of diabetes without having any effect on the secretion of insulin, but exhibiting activity on the metabolic insulin-resistance syndrome.

Its aim is finally to provide a composition which is particularly suitable for diabetics without hyperinsulinism.

These aims and others are achieved by the present invention which relates to a pharmaceutical composition comprising, as active ingredients, metformin optionally in the form of one of its pharmaceutically acceptable salts and a compound of formula (I), in combination with one or more pharmaceutically acceptable excipients.

This composition is particularly appropriate for treating diabetes, more particularly non-insulin-dependent diabetes. It is particularly suitable for reducing hyperglycaemia in non-insulin-dependent diabetes.

The compound of formula (I) is defined in the following manner:

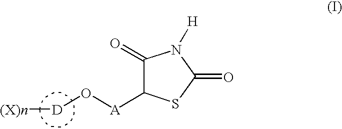

in which A represents a linear or branched, saturated or unsaturated hydrocarbon group comprising from 2 to 16 carbon atoms,

D represents a homo- or heterocarbon-containing mono-, bi- or tricyclic aromatic structure which may include one or more heteroatoms,

X represents a substituent of the aromatic structure, chosen from hydrogen, an alkyl group having from 1 to 6 carbon atoms, an alkoxy group having from 1 to 6 carbon atoms, an alkoxyalkyl group in which the alkoxy and alkyl groups are as defined above, an aryl group defined as an aromatic cyclic structure comprising one or two rings optionally including one or two heteroatoms in the ring, such as for example a phenyl or an α- or β-naphthyl, an arylalkyl group in which the alkyl group is as defined above and the aryl group is as defined above and optionally comprises one or more substituents, an arylalkylaryl group in which the arylalkyl and aryl fractions are as defined above, a halogen, a trifluoromethyl, a cyano, a hydroxyl, a nitro, an amino, a carboxyl, an alkoxycarbonyl, a carboxamide, a sulfonyl, a sulfone, a sulfonamide, a sulfamoyl, an alkylsulfonylamino, an acylamino, a trifluoromethoxy,

n is an integer ranging from 1 to 3,

with the restriction that if A represents a butyl radical,

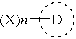

does not represent a 4-chlorophenyl group.

In the preceding text, among the aromatic radicals D, there may be mentioned as homocarbonyl-containing structure the phenyl, α-naphthyl, β-naphthyl, anthracene or fluorenyl radical. Among the heterocyclic aromatic radicals, there may be mentioned pyridyl, or the quinolinyl or carbazolyl ring.

D preferably represents a phenyl or naphthyl radical.

Among the alkyl groups having from 1 to 6 carbon atoms, there may be mentioned in particular a methyl, ethyl, propyl, isopropyl, butyl, isobutyl, tert-butyl, pentyl or hexyl radical. Among the alkoxy groups having from 1 to 6 carbon atoms, there may be mentioned a methoxy, ethoxy, propoxy, isopropoxy, butoxy or isobutoxy radical. Among the halogen groups, there may be mentioned in particular fluorine, chlorine, bromine or iodine.

The chain A is a linear or branched hydrocarbon chain having from 2 to 16 carbon atoms, which is saturated or which has one or more ethylenic unsaturations, optionally substituted with at least one hydroxyl radical or with a phenyl radical. As examples of a linear alkyl radical, there may be mentioned in particular a divalent ethyl, propyl, butyl, pentyl, hexyl, octyl, nonyl, decyl, dodecyl or hexadecyl radical. Among the branched alkyl chains, there may be mentioned in particular the divalent 2-ethylhexyl, 2-methylbutyl, 2-methylpentyl, 1-methylhexyl or 3-methylheptyl radical. Among the monohydroxyalkyl chains, the radicals having 2 or 3 carbon atoms, such as 2-hydroxyethyl, 2-hydroxypropyl or 3-hydroxypropyl, are preferred. Among the polyhydroxyalkyl chains, the radicals having 3 to 6 carbon atoms and 2 to 5 hydroxyl radicals, such as 2,3-dihydroxypropyl, 2,3,4-trihydroxybutyl or 2,3,4,5-tetrahydroxypentyl or the pentaerythritol residue, are preferred. Among the hydrocarbon chains having from 2 to 16 carbon atoms and having one or more ethylenic unsaturations, the divalent allyl radical may be mentioned in particular.

The divalent ethyl or propyl radical is preferred.

The present invention also relates to the tautomeric forms of the compounds of general formula (I), to the enantiomers, diastereoisomers and epimers of these compounds, and to their solvates.

It is possible to envisage that the ketone functional groups carried by the thiazolidine ring can become enolized and give rise to monoenols.

The thiazolidinedione derivatives may, in this case, be salified and may exist in the form of basic salts.

Examples of basic salts of the compounds of general formula (I) include pharmacologically acceptable salts, such as sodium salts, potassium salts, magnesium salts, calcium salts, amine salts and other salts of the same type (aluminium, iron, bismuth, and the like). The amine salts which are not pharmacologically acceptable may serve as a means of identification, purification or resolution.

Among the compounds of general formula (I) according to the invention, there may be mentioned more particularly as compounds which are currently preferred:

5-[3-(4-fluorophenoxy)propyl]thiazolidine-2,4-dione

5-(2-phenoxyethyl)thiazolidine-2,4-dione

5-[2-(4-fluorophenoxy)ethyl]thiazolidine-2,4-dione

5-{[1-hydroxy-2-(4-fluorophenoxy)]ethyl}thiazolidine-2,4-dione

5-{[2-hydroxy-3-(4-fluorophenoxy)]propyl}thiazolidine-2,4-dione

5-[1-methyl-2-phenoxyethyl]thiazolidine-2,4-dione

5-[2-(4-cyanophenoxy)ethyl]thiazolidine-2,4-dione (CRE 16336)

5-[2-(2-fluorophenoxy)ethyl]thiazolidine-2,4-dione

5-[2-(2-naphthyloxy)ethyl]thiazolidine-2,4-dione and their pharmacologically acceptable salts.

These compounds have been described in patent application WO 97/47612.

It is preferable to use 5-[2-(4-cyanophenoxy)ethyl]thiazolidine-2,4-dione (CRE 16336).

According to the invention, metformin or 1,1-dimethylbiguanide may be administered in the form of one of its pharmaceutically acceptable salts such as the hydrochloride, acetate, benzoate, citrate, fumarate, embonate, chlorophenoxyacetate, glycolate, palmoate, aspartate, methanesulfonate, maleate, para-chlorophenoxyisobutyrate, formate, lactate, succinate, sulfate, tartrate, cyclohexanecarboxylate, hexanoate, octanoate, decanoate, hexadecanoate, octadecanoate, benzenesulfonate, trimethoxybenzoate, para-toluenesulfonate, adamantanecarboxylate, glycoxylate, glutamate, pyrrolidonecarboxylate, naphthalenesulfonate, glucose-1-phosphate, nitrate, sulfite, dithionate, phosphate, dobesilate, thioctate, hippurate, 3-benzamidopropanoate, glucuronate, L-pyrrolidone-5-carboxylate, cholate, α-glucose-1-phosphate, alginate, 4-aminobenzoate and the salt with chondriotinsulfuric acid.

Among these salts, the hydrochloride, fumarate, embonate and chlorophenoxyacetate are more particularly preferred.

The pharmaceutically acceptable salts of metformin are obtained in a manner known per se by the action of metformin with the corresponding acid.

The compositions of the invention contain therapeutically effective quantities of the various active ingredients. The ratios of the respective quantities of metformin and the compound of formula (I) therefore vary as a consequence.

Preferably, the weight ratio of metformin or of its pharmaceutically acceptable salt to the compound of formula (I) varies from 1/1, preferably 40/1, better still 2/1 to 20/1.

The compositions of the invention are preferably administered parenterally, or better still orally, other routes of administration not however being excluded, such as for example rectal administration.

When oral administration is envisaged, the compositions of the invention exist in the form of gelatin capsules, effervescent tablets, uncoated or coated tablets, sachets, sugar-coated tablets, oral solutions or ampoules, microgranules or prolonged-release forms.

When parenteral administration is envisaged, the compositions of the invention exist in the form of solutions and suspensions for injection packaged in ampoules or vials for slow venous infusion.

The forms for oral administration are prepared by mixing the active substance with various types of excipient or vehicle such as fillers, disintegrating agents, binders, colourings, flavour correctors and the like, followed by the forming of the mixture.

The colouring may be any of those authorized for a galenic use.

Examples of flavour correctors include cocoa powder, mint, borneol and powdered cinnamon.

As examples of binders, there may be mentioned polyvinylpyrrolidone, hydroxypropyl methyl cellulose, alginic acid, carbomer, carboxymethyl cellulose, dextrin, ethyl cellulose, starch, sodium alginate, polymethacrylate, maltodextrin, liquid glucose, magnesium and aluminium silicate, hydroxyethyl cellulose, ethyl cellulose, methyl cellulose and guar gum.

As disintegrating agent, it is possible to use alginic acid, sodium carboxymethyl cellulose, colloidal silicon dioxide, sodium croscarmellose, crospovidone, guar gum, magnesium and aluminium silicate, methyl cellulose, microcrystalline cellulose, potassium polacrinlin, powdered cellulose, pregelatinized starch, sodium alginate and glycolate of starch and sodium.

Fillers are for example cellulose, lactose, calcium hydrogen phosphate and microcrystalline cellulose.

The tablets may be obtained in a conventional manner by compression of granules in the presence of one or more lubricants. Appropriate lubricants are calcium stearate, glyceryl monostearate, glyceryl palmitostearate, hydrogenated castor oil, hydrogenated vegetable oil, low-fat mineral oil, magnesium stearate, polyethylene glycol, sodium benzoate, sodium lauryl sulfate, sodium stearyl fumarate, stearic acid, talc and zinc stearate. These tablets may then be coated with polymers in solution or suspension, such as hydroxypropyl methyl cellulose or ethyl cellulose.

The granules used to do this are for example prepared using the wet granulation method from a mixture of the active ingredients with one or more excipients such as a binder, a disintegrating agent and a filler.

To obtain hard capsules, the mixture of active ingredients with an appropriate filler (for example lactose) is incorporated into empty gelatin capsules optionally in the presence of a lubricating agent such as magnesium stearate, stearic acid, talc or zinc stearate.

Soft capsules or gelatin capsules are prepared by solubilizing the active ingredients in an appropriate solvent (for example polyethylene glycol) followed by incorporation into soft capsules.

The forms for parenteral administration are obtained in a conventional manner by mixing active ingredients with buffers, stabilizing agents, preservatives, solubilizing agents, isotonizing agents and suspending agents. In accordance with known techniques, these mixtures are then sterilized and then packaged in the form of intravenous injections.

As a buffer, persons skilled in the art may use buffers based on organic phosphate salts.

Examples of suspending agents include methyl cellulose, hydroxyethyl cellulose, acacia and sodium carboxymethyl cellulose.

Examples of a solubilizing agent include castor oil solidified with polyoxyethylene, polysorbate 80, nicotinamide or macrogol.

In addition, useful stabilizers according to the invention are sodium sulfite and sodium metasulfite, while there may be mentioned sodium p-hydroxybenzoate, sorbic acid, cresol and chlorocresol as preservatives. For the preparation of an oral solution or suspension, the active ingredients are dissolved or suspended in an appropriate vehicle with a dispersing agent, a humectant, a suspending agent (for example polyvinylpyrrolidone), a preservative (such as methylparaben or propylparaben), a flavour corrector or a colouring.

For the preparation of suppositories, the active ingredients are mixed in a manner known per se with an appropriate base constituent such as polyethylene glycol or semisynthetic glycerides.

For the preparation of microcapsules, the active ingredients are combined with appropriate diluents, appropriate stabilizers, agents promoting prolonged release of the active substances or any other type of additive for the formation of a central core which is then coated with an appropriate polymer (for example a water-soluble resin or a water-insoluble resin). Techniques known to persons skilled in the art will be used to this end.

The microcapsules thus obtained are then optionally formulated in appropriate dosage units.

The subject of the present invention is also the use of metformin optionally in the form of one of its pharmaceutically acceptable salts in combination with a compound of formula (I) as defined above for the preparation of a medicinal combination intended for treating diabetes, more particularly non-insulin-dependent diabetes.

According to another of its aspects, the invention relates to the use of metformin optionally in the form of one of its pharmaceutically acceptable salts in combination with the said compound of formula (I) for the preparation of a medicinal combination intended for reducing hyperglycaemia in non-insulin-dependent diabetes.

The subject of the present invention is also a method for treating diabetes, more particularly non-insulin-dependent diabetes, in a mammal, comprising administering to the said mammal the composition according to the present invention.

Mefformin may be provided in the form of any of the salts defined above; however, the use of metformin as it is or in the hydrochloride, fumarate, embonate or chlorophenoxyacetate form is preferred.

When metformin or its salt and the compound of formula (I) are incorporated into the same unit dose, the unit dose preferably comprises from 50 to 1000 mg of metformin.

Advantageously, the unit dose comprises, in this case, from 12.5 to 50 mg of a compound of formula (I).

The dosage naturally depends on the mode of administration, the therapeutic indication, the age of the patient and their condition.

In general, the daily dosage varies between 100 and 2000 mg of metformin and between 25 and 100 mg of compound of formula (I).

Concrete but nonlimiting examples of the invention will now be presented.

### EXAMPLE 1

A tablet having the following composition is prepared:

| | | |
|---|---|---|
| Metformin | 850 mg | 77.3% |
| 5-[2-(4-Cyanophenoxy)ethyl]thiazolidine- | 50 mg | 4.5% |
| 2,4-dione | | |
| Lactose | 99 mg | 9% |
| Hydroxypropyl cellulose | 35 mg | 3.2% |
| Sodium croscarmellose | 55 mg | 5% |
| Magnesium stearate | 11 mg | 1% |

### EXAMPLE 2

A tablet having the following composition is prepared:

| | | | |
|---|---|---|---|
| Metformin | 500 | mg | 80% |
| 5-[2-(4-Cyanophenoxy)ethyl]thiazolidine- | 25 | mg | 4% |
| 2,4-dione | | | |
| Lactose | 37.5 | mg | 6% |
| Hydroxypropyl cellulose | 25 | mg | 4% |
| Sodium croscarmellose | 31.25 | mg | 5% |
| Magnesium stearate | 6.25 | mg | 1% |

### Cre 16336 and Metformin Combination Test for a Beneficial Effect of this Combination in the Treatment of Hyperglycaemia

Pharmacological Study

The antidiabetic effect of the metformin and CRE16336 combination was studied on the nOSTZ rat, an experimental model of non-insulin-dependent diabetes.

This model is produced by intravenous injection of Streptozotocin (STZ) 100 mg/kg, on the day of birth.

The characteristics of this model are:

a hyperglycaemia

an absence of basal hypoinsulinaemia

a glucose intolerance

an absence of insulin resistance

Experimental Protocol

38 male nOSTZ rats were used after selection based on the hyperglycaemia value after fasting for 2 h in order to homogenize the groups.

They were then divided into 4 groups:

a control nOSTZ group

a group treated with metformin at 25 mg/kg

a group treated with CRE16336 at 12.5 mg/kg

a group treated with metformin 25 mg/kg and CRE16336 12.5 mg/kg

5 male Wistar rats were incorporated into this study in order to evaluate the degree of hyperglycaemia in the diabetic animals.

The products were administered orally in the morning between 8 am and 9 am, for 4 days. The glycaemia, insulinaemia and lacticaemia were determined, after 4 days of treatment, by collecting blood samples from the tail of the rats anaesthesized beforehand, 2 h after the last administration of the products.

Results

| | T0 | 4 DAYS | | |
|---|---|---|---|---|
| | Glucose | Glucose | Lactate | Insulin |
| | mg/dl | mg/dl | mg/dl | μU/ml |
| Control nOSTZ | 221 ± 5 | 169 ± 6 | 11.5 ± 1 | 37.0 ± 7.3 |
| Metformin 25 mg/kg | 219 ± 4 | 160 ± 5 | 15.4 ± 1.4 | 34.1 ± 3.7 |
| CRE16336 12.5 mg/kg | 219 ± 4 | 156 ± 6 | 13.2 ± 0.7 | 45.2 ± 5.5 |
| Metformin 25 mg/kg + | 221 ± 4 | 138 ± 6 | 14.7 ± 0.5 | 44.4 ± 4.3 |
| CRE16336 12.5 | | | | |
| mg/kg | | | | |
| Control Wistar | 137 ± 1 | 134 ± 5 | 14.3 ± 0.5 | 21.3 ± 3.6 |

Comments

After 4 days of treatment (placebo), the glycaemia in the control nOSTZ rats reduces because of a “nursing” effect. The hyperglycaemia is however still present in these animals 169±6 vs 134±5 mg/dl in the Wistar rats.

The treatment with metformin or CRE16336 in very low dose does not modify the hyperglycaemia in the nOSTZ rats.

Unlike the monotherapy treatments, the combination of metformin and CRE16336, administered at ineffective doses, induces a significant reduction in the hyperglycaemia which regresses from 31 mg/dl (138±63 mg/dl vs 169±6 mg/dl in the control group).

The metformin and CRE16336 combination brings about normalization of the glycaemia at doses where, given separately, these 2 products are without effect on the hyperglycaemia.

1. Pharmaceutical composition comprising, as active ingredients, (i) metformin optionally in the form of one of its pharmaceutically acceptable salts and (ii) a compound of formula (I) in combination with one or more pharmaceutically acceptable excipients, the compound of formula (I) being defined in the following manner: 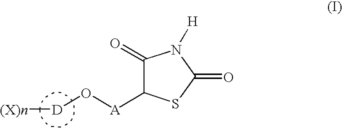
in which A represents a linear or branched, saturated or unsaturated hydrocarbon group comprising from 2 to 16 carbon atoms,
D represents a homo- or heterocarbon-containing mono-, bi- or tricyclic aromatic structure which may include one or more heteroatoms,
X represents a substituent of the aromatic structure, chosen from hydrogen, an alkyl group having from 1 to 6 carbon atoms, an alkoxy group having from 1 to 6 carbon atoms, an alkoxyalkyl group in which the alkoxy and alkyl groups are as defined above, an aryl group defined as an aromatic cyclic structure comprising one or two rings optionally including one or two heteroatoms in the ring, such as for example a phenyl or an α- or β-naphthyl, an arylalkyl group in which the alkyl group is as defined above and the aryl group is as defined above and optionally comprises one or more substituents, an arylalkylaryl group in which the arylalkyl and aryl fractions are as defined above, a halogen, a trifluoromethyl, a cyano, a hydroxyl, a nitro, an amino, a carboxyl, an alkoxycarbonyl, a carboxamide, a sulfonyl, a sulfone, a sulfonamide, a sulfamoyl, an alkylsulfonylamino, an acylamino, a trifluoromethoxy,
n is an integer ranging from 1 to 3,
with the restriction that if A represents a butyl radical, 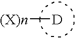
does not represent a 4-chlorophenyl group.

2. Composition according to claim 1, for treating diabetes.

3. Composition according to either of claims 1 and 2, for treating non-insulin-dependent diabetes.

4. Pharmaceutical composition according to any one of claims 1 to 3, characterized in that the weight ratio of metformin or of its pharmaceutically acceptable salt to the compound of formula (I) varies from 1/1 to 40/1.

5. Pharmaceutical composition according to any one of the preceding claims, characterized in that the metformin salt is a hydrochloride, fumarate, embonate or chlorophenoxyacetate.

6. Composition according to any one of the preceding claims, characterized in that the compound of formula (I) is chosen from:
5-[3-(4-fluorophenoxy)propyl]thiazolidine-2,4-dione
5-(2-phenoxyethyl)thiazolidine-2,4-dione
5-[2-(4-fluorophenoxy)ethyl]thiazolidine-2,4-dione
5-{[1-hydroxy-2-(4-fluorophenoxy)]ethyl}thiazolidine-2,4-dione
5-{[2-hydroxy-3-(4-fluorophenoxy)]propyl}thiazolidine-2,4-dione
5-[1-methyl-2-phenoxyethyl]thiazolidine-2,4-dione
5-[2-(4-cyanophenoxy)ethyl]thiazolidine-2,4-dione
5-[2-(2-fluorophenoxy)ethyl]thiazolidine-2,4-dione
5-[2-(2-naphthyloxy)ethyl]thiazolidine-2,4-dione and their pharmacologically acceptable salts.

7. Composition according to claim 6, characterized in that the compound of formula I is 5-[2-(4-cyanophenoxy)ethyl]thiazolidine-2,4-dione.

8. Composition according to any one of the preceding claims, which is appropriate for oral administration.

9. Use of metformin optionally in the form of one of its pharmaceutically acceptable salts in combination with a compound of formula (I) as defined in claim 1, for the preparation of a medicinal combination intended for treating diabetes.

10. Use according to claim 9, for the preparation of a medicinal combination intended for treating non-insulin-dependent diabetes.

11. Use according to either of claims 9 and 10, characterized in that the metformin salt is a hydrochloride, a fumarate, an embonate or a chlorophenoxyacetate.

12. Use according to one of claims 9 to 11, characterized in that the compound of formula (I) is chosen from:
5-[3-(4-fluorophenoxy)propyl]thiazolidine-2,4-dione
5-(2-phenoxyethyl)thiazolidine-2,4-dione
5-[2-(4-fluorophenoxy)ethyl]thiazolidine-2,4-dione
5-{[1-hydroxy-2-(4-fluorophenoxy)]ethyl}thiazolidine-2,4-dione
5-{[2-hydroxy-3-(4-fluorophenoxy)]propyl}thiazolidine-2,4-dione
5-[1-methyl-2-phenoxyethyl]thiazolidine-2,4-dione
5-[2-(4-cyanophenoxy)ethyl]thiazolidine-2,4-dione
5-[2-(2-fluorophenoxy)ethyl]thiazolidine-2,4-dione
5-[2-(2-naphthyloxy)ethyl]thiazolidine-2,4-dione and their pharmacologically acceptable salts.

13. Use according to any one of claims 9 to 12, characterized in that the medicinal combination is provided in the form of a unit dose containing metformin or one of its pharmaceutically acceptable salts, and a compound of formula (I).

14. Use according to claim 13, characterized in that the unit dose comprises from 50 to 1000 mg of metformin and from 12.5 to 50 mg of a compound of formula (I).
